(19) United States
(12) Plant Patent Application Publication
(10) Pub. No.: US 2021/0321549 P1
(43) Pub. Date: Oct. 14, 2021

(54) Ipomoea batatas plant named 'Churakanasa'

(50) ;

(71) Applicant: National Agriculture and Food Research Organization, Ibaraki (JP)

(72) Inventors: Akira KOBAYASHI, Ibaraki (JP); Yumi KAI, Ibaraki (JP); Takeo SAKAIGAICHI, Ibaraki (JP); Keisuke SUEMATSU, Ibaraki (JP); Katsuya ICHINOSE, Ibaraki (JP); Yoshihiro OKADA, Ibaraki (JP)

(21) Appl. No.: 17/300,123

(22) Filed: Mar. 15, 2021

(30) Foreign Application Priority Data
Mar. 17, 2020 (JP) 34567

(51) Int. Cl. A01H 6/00

(52) U.S. Cl. PLT/258

(57) ABSTRACT

‘Churakanasa’ is a new variety of sweet potato bred by hybridizing variety ‘Purple Sweet Lord’ and ‘90IDN-47’ in 2009. The plant may be used, e.g., for cultivation of root tubers for consumption.

### CROSS REFERENCE TO RELATED APPLICATION

The present application claims priority to Japanese Plant Variety Registration No. 34567 filed Mar. 17, 2020, the entire contents of which is incorporated herein for all purposes by this reference.

### PLANT NAME AND VARIETY DENOMINATION

Latin name of the genus and species of the plant claimed: Ipomoea batatas (L.) Lam. The claimed plant is a hybridization of different varieties of Ipomoea batatas.

Common name of the claimed plant's species: sweet potato.

Variety denomination: ‘Churakanasa’.

### BACKGROUND

Ipomoea batatas, commonly called sweet potato or sweet potato vine, is a dicotyledonous plant that belongs to the bindweed or morning glory family, Convolvulaceae. It is an herbaceous perennial that is cultivated for its edible root tubers. Ipomoea batatas is native to tropical regions of the Americas.

Because sweet potatoes are very strong against climate disasters, such as typhoons and droughts, sweet potatoes are significant crops in Okinawa. Purple colored sweet potato produced in Okinawa is specifically called ‘Beniimo’ which means red sweet potato. Processed paste confectionaries which utilize the color tone of purple colored sweet potato are popular as souvenirs.

### BRIEF SUMMARY

‘Churakanasa’ is a new variety of Ipomoea batatas bred by hybridization of variety ‘Purple Sweet Lord’ (patented) as the female parent and ‘90IDN-47’ (not patented; genetic resource of Indonesia) as the male parent. The plant may be used, e.g., for cultivation of root tubers for consumption.

‘Churakanasa’ is a purple-colored sweet potato lineage. The weight of the yield of good quality storage roots of ‘Churakanasa’ is comparable to that of the high yield variety ‘Churakoibeni’ in conventional spring planting. In autumn planting cultivation, the yield (total weight) of the good quality storage roots of ‘Churakanasa’ is greater than that of ‘Churakoibeni,’ thus the introduction of autumn planting cultivation of ‘Churakanasa’ is possible. The color value representing the anthocyanin content of ‘Churakanasa’ is moderately higher than that of ‘Churakoibeni.’ The color tone of the flesh paste is moderately more bluish than that of ‘Churakoibeni,’ but the processability of ‘Churakanasa’ into paste and red sweet potato tart was evaluated to be the same level as that of ‘Churakoibeni.’

### BRIEF DESCRIPTION OF THE DRAWINGS

FIG. 1 is a photograph of the above-ground or aerial portions of ‘Churakanasa’ (top) compared to similar varieties ‘Ayamurasaki’ (bottom left) and ‘Murasakimasari’ (bottom right).

FIG. 2 is a photograph of the below-ground or subterranean portions of ‘Churakanasa’ (top) compared to similar varieties ‘Ayamurasaki’ (bottom left) and ‘Murasakimasari’ (bottom right).

FIG. 3 is a photograph of a cross-sectional view of the storage roots or subterranean portions of ‘Churakanasa’ (top) compared to similar varieties ‘Ayamurasaki’ (middle) and ‘Murasakimasari’ (bottom) showing the colors of the storage roots.

FIG. 4 is a photograph of the whole plant body of ‘Churakanasa’ (left) compared to the similar variety ‘Ayamurasaki’ (right).

FIG. 5 is a photograph of the cultivation zone of ‘Churakanasa.’

FIG. 6 is a photograph of the paste produced from ‘Churakanasa’ (right) and similar variety ‘Churakoibeni’ (left).

FIG. 7 is a photograph of Beniimo (red sweet potato) tarts prepared using paste from ‘Churakanasa’ (right) and similar variety ‘Churakoibeni’ (left).

FIG. 8 is a graph showing weevil damage rate and damage degree for ‘Churakanasa’ as well as varieties ‘Shiroyutaka,’ ‘Tamayutaka,’ ‘Churakoibeni,’ and ‘Murasakihomare.’

FIG. 9 is a graph showing storage root weight and number for Thurakanasa' as well as varieties ‘Churakoibeni,’ ‘Murasakihomare,’ ‘Tamayutaka,’ and ‘Shiroyutaka.’

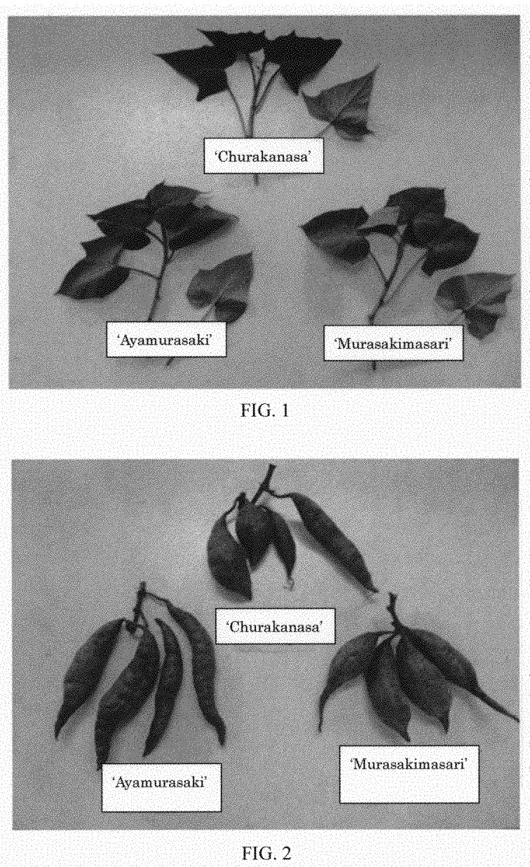

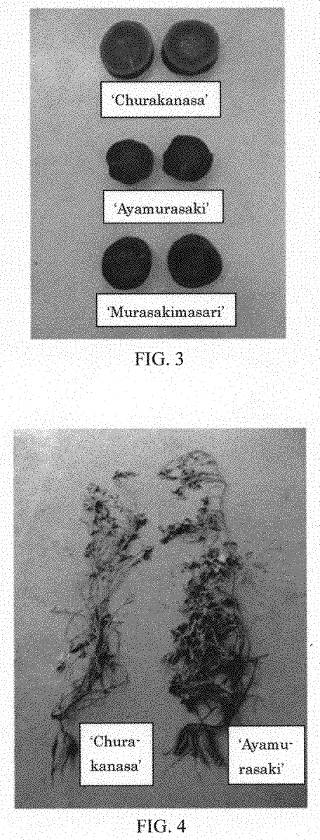

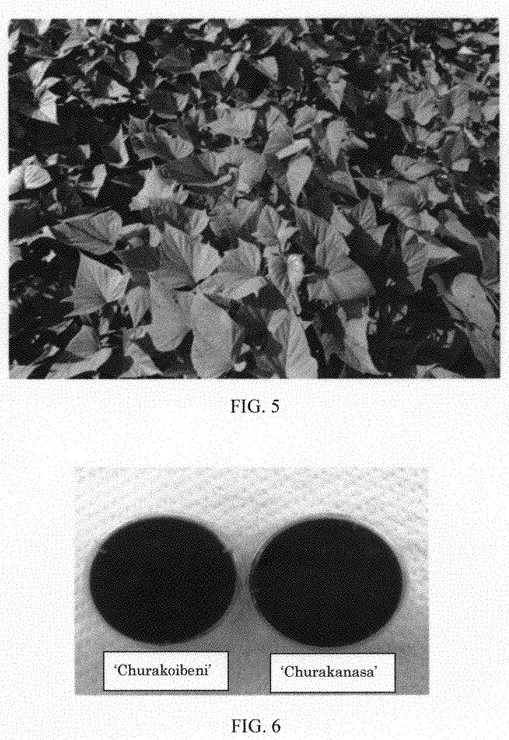

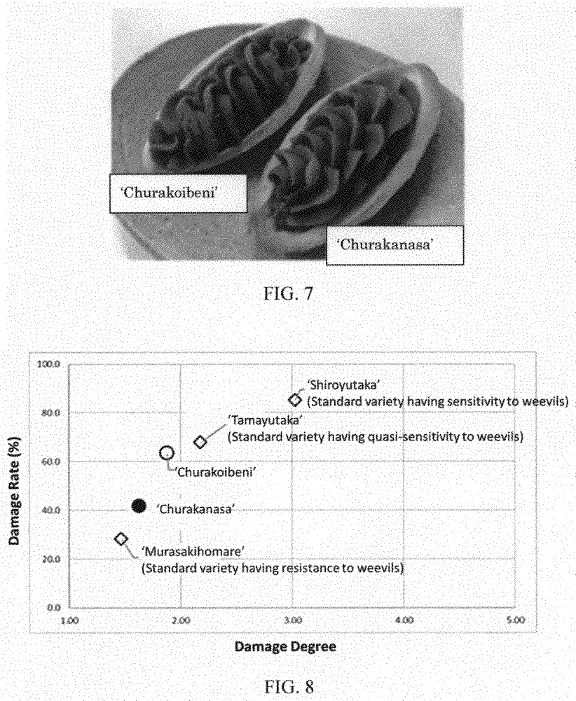

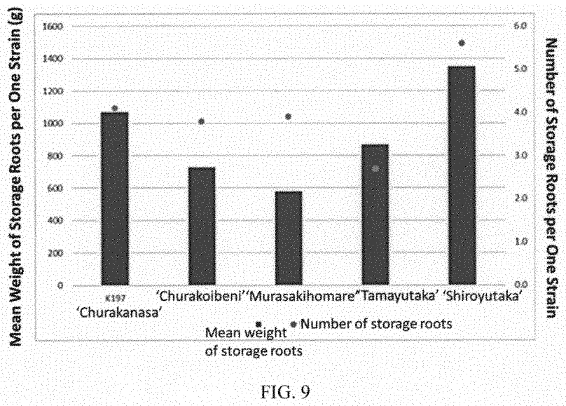

### DETAILED BOTANICAL DESCRIPTION OF THE PLANT

Variety: ‘Churakanasa’

Species of the plant claimed: Ipomoea batatas (L.) Lam. The claimed plant is bred by hybridizing variety ‘Purple Sweet Lord’ (patented) as the female parent and Indonesian native lineage ‘90IDN-47’ (not patented) as the male parent.

Common names of the claimed plant's species include: sweet potato.

Characteristics of ‘Churakanasa’ provided herein were observed when the plant was cultivated at Yokoichi-machi, Miyazaki, Japan in open culture.

Sweet potato ‘Churakanasa’ is a lineage selected from a mating combination in which ‘Purple sweet lord’ having a purple color flesh and excellent appearance is used as a mother, and Indonesian native lineage, ‘90IDN-47’ having soil rot resistance is used as a father.

Cross-breeding and collecting seeds were carried out in 2009. Selection and breeding were carried out at Okinawa Region from 2011 onwards. Based on the result of seedling individual selection test in 2011, a lineage was selected which showed dark purple flesh color and was excellent in appearance and root tuberization, and was given the lineage number of ‘Kyukei 09307-72’. Thereafter, ‘Kyukei 09307-72’ was used for the lineage selection preliminary test in 2012, the lineage selection test in 2013, the productivity check preliminary test in 2014, and the productivity check test in 2015. As a result, the yield of spring cultivation of the selected lineage in Okinawa is almost the same as that of ‘Churakoibeni’, and the selected lineage is excellent in resistance to soil rot, etc., and therefore, the system number of ‘Kyukei 348’ was assigned to the selected lineage in 2017. From 2017 onwards, in addition to spring cultivation, the yield was evaluated in the autumn cultivation, and the processability was evaluated by the actual users. From 2018 onwards, black rot disease (Ceratocystis fimbriata) resistance test and soil rot resistance test have been carried out. As a result of examining various characteristics, the system name of ‘Kyushu No. 197’ was assigned to the finally selected lineage in December 2018. ‘Kyushu No. 197’ was named ‘Churakanasa.’ Incidentally, the field productivity test was carried out in Okinawa in 2016-2018.

Morphological Characteristics

The plant growth habit in a field is the ‘spreading’ type. The length of primary shoots of the stem is ‘medium.’ (131.8 cm) The length of internode of the stem is ‘medium.’ (6.7 cm) The diameter of internode of the stem is ‘medium to large.’ (7.06 mm) The anthocyanin coloration of internode of stem, tip of stem, and node of stem are ‘medium’, ‘medium’ and ‘medium’ respectively; and the pubescence of tip is ‘dense’. The shape of the leaf blade is the ‘Single incision lobed’ type in Seed and Seedling Characteristics Classification Survey References (the old standard on March, 1981). The size of the leaf blade is ‘medium’ (215.2 cm²); the number of lobes of the leaf blade is ‘3.’ The depth of lobing of the leaf blade is ‘moderate.’ The color and the anthocyanin coloration on upper side of the leaf blade are ‘green’ and ‘weak’, respectively. The extent and the intensity of anthocyanin coloration on abaxial veins on lower side of the leaf blade are ‘medium’ and ‘weak’, respectively. The anthocyanin coloration of nectary is ‘strong’. The main color on upper side and lower side of the young leaf blade are ‘purplish brown’ and ‘light purple’, respectively. The anthocyanin coloration of the petiole is ‘weak’, and the length of the petiole is ‘medium’. (25.3 cm) The flowering in open-field culture is ‘absent’.

The strength of the storage root's neck is ‘medium.’ The position of the storage root (same as depth from the field surface to storage root) is ‘medium’, and the difficulty of digging (harvesting) the storage root (same as root tuber) is ‘medium’. The shape of storage root is ‘elliptic’. The uniformity in shape of storage roots is ‘medium to low.’ The size of storage roots is ‘small to medium’, (142 grams) and the uniformity in size of storage roots is ‘medium to low’. The colors of skin and flesh of the storage root are ‘light purple’ and ‘purple’, respectively. The depth of eyes of the storage root is ‘medium’ as measured using visual observation. The skin roughness of the storage root is ‘medium.’ The grooves, dehiscence and ridges of the storage root are all ‘absent’ and the appearance is ‘medium’.

Ecological characteristics

The timing (early or late) of sprouting from storage root is ‘late.’ The uniformity in time of sprouting from different storage roots (almost simultaneous or not) is ‘medium to high.’ The speed of sprout elongation is ‘slow’, and the number of sprouts is ‘medium’, as measured using visual observation, and the sprouting trait (same as sprouting habit) is ‘poor’.

Under the standard cultivation at the breeding place, the yield (total weight) of the good quality storage roots is lower than those of ‘Ayamurasaki ’ and ‘Murasakimasari’. The average yield for ‘Churakanasa’ is 183 kg/a, while the average yield for ‘Murasakimasari’ is 329 kg/a and average yield for ‘Ayamurasaki’ is 307 kg/a. Also, the percentage of dry matter content and the starch content are lower than those of ‘Ayamurasaki ’ and ‘Murasakimasari’.

The resistance to Southern root-knot nematode (M. incognita) is ‘medium’ and the resistance to Minami root-lesion nematodes (P. coffeae) is ‘moderately strong’. The resistance to black rot disease (Ceratocystis fimbriata) is ‘medium.’ The resistance to soil rot is ‘moderately strong’, and the resistance to stem rot (Fusarium axysporum f, sp. batatas) is ‘medium’. Storability of storage root is ‘moderately difficult’.

Quality characteristics

The taste of steamed storage root is ‘medium’, and exceeds that of ‘Ayamurasaki ’ and ‘Murasakimasari’. The color value indicating the amount of anthocyanin content is a little higher than that of ‘Churakoibeni’, and lower than that of ‘Ayamurasaki’ and ‘Murasakimasari’. The color of paste made from the flesh is a little more bluish than that of ‘Ayamurasaki ’ and ‘Murasakimasari’.

Varieties that are similar to ‘Churakanasa’ are ‘Murasakimasari’ and ‘Ayamurasaki.’ Characteristics of ‘Murasakimasari’ and ‘Ayamurasaki’ are compared in Tables 1 through 3 below.

TABLE 1
Distinguishable characteristics between ‘Churakanasa’and similar varieties

| Denomination | | State of | State of |
|---|---|---|---|
| of Similar | Description of | Expression in | Expression in |
| Variety | Characteristics | Similar Variety | Candidate Variety |
| Murasakimasan | Young leaf blade: main | light green | purplish brown |
| | color on upper side | | |
| Ayamurasaki | Leaf blade: extent of | absent or very | medium |
| | anthocyanin coloration | small | |
| | on abaxial veins on | | |
| | lower side | | |
| Ayamurasaki | Leaf blade: intensity of | very weak | weak |
| | anthocyanin coloration | | |
| | on abaxial veins on | | |
| | lower side | | |

TABLE 2
Description of physical characteristics of ‘Churakanasa’ and values for
characteristics of similar varieties for year 2018

| | | | Class values of candidate variety (Comparison with | | | | | | |
|---|---|---|---|---|---|---|---|---|---|
| Characteristics | UPOV | | Ex.Var.(standard varieties)) ‘CHURAKANASA’ | | | | | | |
| No. | No. | Characteristics | 01 | 02 | 03 | 04 | 05 | 06 | 07 |
| 1 | 1 | Plant: | upright | | semi- | | spreading | | |
| | | growth | | | upright | | | | |
| | | habit | | | | | | | |
| 2 | 2 | Stem: | | | short | Short | medium | | long |
| | | length | | | | to | | | |
| | | of | | | | medium | | | |
| | | primary | | | | | | | |
| | | shoots | | | | | | | |
| 3 | 3 | Stem: | | | short | | medium | | long |
| | | length | | | | | | | |
| | | of | | | | | | | |
| | | inter | | | | | | | |
| | | node | | | | | | | |
| 4 | 4 | Stem: | | | small | | medium | | large |
| | | diameter | | | | | | | |
| | | of | | | | | | | |
| | | inter | | | | | | | |
| | | node | | | | | | | |
| 5 | 5 | Stem: | absent | weak | medium | strong | | | |
| | | anthocyanin | or | | | | | | |
| | | coloration | very | | | | | | |
| | | of | weak | | | | | | |
| | | inter | | | | | | | |
| | | node | | | | | | | |
| 6 | 6 | Stem: | absent | weak | medium | strong | | | |
| | | anthocyanin | or | | | | | | |
| | | coloration | very | | | | | | |
| | | of tip | weak | | | | | | |
| 7 | 7 | Stem: | absent | weak | medium | strong | | | |
| | | anthocyanin | or | | | | | | |
| | | coloration | very | | | | | | |
| | | of inter | weak | | | | | | |
| | | node | | | | | | | |
| | | and | | | | | | | |
| | | lateral | | | | | | | |
| | | branch | | | | | | | |
| 8 | 8 | Stem: | absent | sparce | medium | dense | | | |
| | | pubescence | or | | | | | | |
| | | of tip | very | | | | | | |
| | | | sparce | | | | | | |
| 9 | | Leaf | | | small | small | medium | | large |
| | | blade: | | | | to | | | |
| | | size | | | | medium | | | |
| 10 | 9 | Leaf | absent | Three | five | seven | | | |
| | | blade: | | lobes | lobes | lobes | | | |
| | | number | | | | | | | |
| | | of lobes | | | | | | | |
| 12 | 11 | Only | | | shallow | | moderate | | deep |
| | | varieties | | | | | | | |
| | | with | | | | | | | |
| | | leaf | | | | | | | |
| | | blade | | | | | | | |
| | | lobes: | | | | | | | |
| | | Leaf | | | | | | | |
| | | blade: | | | | | | | |
| | | depth | | | | | | | |
| | | of | | | | | | | |
| | | lobing | | | | | | | |
| 13 | 12 | Excluding | yellow | green | grey | | | | |
| | | ornamental | green | | green | | | | |
| | | variety: | | | | | | | |
| | | Leaf | | | | | | | |
| | | blade: | | | | | | | |
| | | color | | | | | | | |
| | | on | | | | | | | |
| | | upper | | | | | | | |
| | | side | | | | | | | |
| | | (excluding | | | | | | | |
| | | anthocyanin | | | | | | | |
| | | coloration) | | | | | | | |
| 19 | 13 | Excluding | absent | weak | medium | strong | | | |
| | | ornamental | or | | | | | | |
| | | variety: | very | | | | | | |
| | | Leaf | weak | | | | | | |
| | | blade: | | | | | | | |
| | | anthocyanin | | | | | | | |
| | | coloration | | | | | | | |
| | | on upper | | | | | | | |
| | | side | | | | | | | |
| 20 | 14 | Leaf | absent | | small | | medium | | large |
| | | blade: | or | | | | | | |
| | | extent of | very | | | | | | |
| | | anthocyanin | small | | | | | | |
| | | coloration | | | | | | | |
| | | abaxial | | | | | | | |
| | | veins | | | | | | | |
| | | on lower | | | | | | | |
| | | side | | | | | | | |
| 21 | 15 | Leaf | absent | | weak | | medium | | strong |
| | | blade: | or | | | | | | |
| | | intensity of | very | | | | | | |
| | | anthocyanin | weak | | | | | | |
| | | coloration | | | | | | | |
| | | abaxial | | | | | | | |
| | | veins on | | | | | | | |
| | | lower | | | | | | | |
| | | side | | | | | | | |
| 22 | | Leaf | absent | | weak | | medium | | strong |
| | | blade: | or | | | | | | |
| | | anthocyanin | very | | | | | | |
| | | coloration | weak | | | | | | |
| | | of nectary | | | | | | | |
| 23 | 16 | Young | yellow | light | medium | dark | light | medium | Purpleish |
| | | leaf | green | green | green | green | purple | purple | brown |
| | | blade: | | | | | | | |
| | | main | | | | | | | |
| | | color on | | | | | | | |
| | | upper | | | | | | | |
| | | side | | | | | | | |
| 24 | | Young | yellow | light | medium | dark | Light | medium | purpleish |
| | | leaf | green | green | green | green | purple | purple | brown |
| | | blade: | | | | | | | |
| | | main | | | | | | | |
| | | color on | | | | | | | |
| | | lower | | | | | | | |
| | | side | | | | | | | |
| 25 | 17 | Petiole: | absent | | weak | | medium | | strong |
| | | anthocyanin | or | | | | | | |
| | | coloration | very | | | | | | |
| | | | weak | | | | | | |
| 26 | 18 | Petiole: | | | short | | medium | | long |
| | | length | | | | | | | |
| 29 | 19 | Storage | obovate | elliptic | ovate | oblong | irregular | | |
| | | root: | | | | | | | |
| | | shape | | | | | | | |
| 30 | 20 | Storage | | | moderately | | medium | | moderately |
| | | root: | | | compressed | | | | elongated |
| | | ratio | | | | | | | |
| | | length/ | | | | | | | |
| | | width | | | | | | | |
| 31 | | Storage | | | few | | medium | | many |
| | | root: | | | | | | | |
| | | number | | | | | | | |
| 32 | | Storage | | | small | Small | medium | | large |
| | | root: | | | | to | | | |
| | | mean | | | | medium | | | |
| | | weight | | | | | | | |
| 33 | 21 | Storage | | | thin | | medium | | thick |
| | | root: | | | | | | | |
| | | thickness | | | | | | | |
| | | of cortex | | | | | | | |
| | | relative | | | | | | | |
| | | to over | | | | | | | |
| | | all | | | | | | | |
| | | diameter | | | | | | | |
| 34 | 22 | Storage | white | light | yellow | orange | brownish | pink | red |
| | | root: | | beige | | | orange | | |
| | | main | | | | | | | |
| | | color of | | | | | | | |
| | | skin | | | | | | | |
| 35 | 23 | Storage | absent | white | yellow | orange | pink | red | purple |
| | | root: | | | | | | | |
| | | secondary | | | | | | | |
| | | color of | | | | | | | |
| | | skin | | | | | | | |
| 36 | 24 | Storage | white | light | yellow | orange | purple | | |
| | | root: | | beige | | | | | |
| | | main | | | | | | | |
| | | color of | | | | | | | |
| | | flesh | | | | | | | |
| 37 | 25 | Storage | light | medium | dark | | | | |
| | | root: | | | | | | | |
| | | intensity | | | | | | | |
| | | of main | | | | | | | |
| | | color of | | | | | | | |
| | | flesh | | | | | | | |
| 38 | 26 | Storage | white | light | yellow | orange | pink | red | red |
| | | root: | | beige | | | | | purple |
| | | secondary | | | | | | | |
| | | color of | | | | | | | |
| | | flesh | | | | | | | |
| 39 | 27 | Storage | shallow | medium | deep | | | | |
| | | root: | | | | | | | |
| | | depth | | | | | | | |
| | | of eyes | | | | | | | |
| 40 | | Storage | white | gray | light | light | light | yellow | gray |
| | | root: | | white | yellow | beige | yellow | | yellow |
| | | color of | | | white | | | | |
| | | steamed | | | | | | | |
| | | flesh | | | | | | | |
| 44 | | Southern | weak | moderately | medium | moderately | strong | | |
| | | rootknot | | weak | | strong | | | |
| | | nematode | | | | | | | |
| | | (Meloisogyne) | | | | | | | |
| | | resistance | | | | | | | |
| | | | Class values of candidate variety | | | | | | |
| | | | (Comparison with Ex.Var.(standard | | | | Remarks | Class Values of | |
| Characteristics | UPOV | | varieties)) ‘CHURAKANASA’ | | | | (Measurement | most similar varieties | |
| No. | No. | Characteristics | 08 | 09 | 10 | 11 | Value etc.) | ‘Ayamurasaki’ | ‘Murasakimasari’ |
| 1 | 1 | Plant: | | | | | | 5 | 5 |
| | | growth | | | | | | | |
| | | habit | | | | | | | |
| 2 | 2 | Stem: | | | | | 69.0 | 5 | 5 |
| | | length | | | | | cm | (99.9 cm) | (84.2 cm) |
| | | of | | | | | | | |
| | | primary | | | | | | | |
| | | shoots | | | | | | | |
| 3 | 3 | Stem: | | | | | 4.1 | 5 | 5 |
| | | length | | | | | cm | (4.9 cm) | (4.2 cm) |
| | | of | | | | | | | |
| | | inter | | | | | | | |
| | | node | | | | | | | |
| 4 | 4 | Stem: | | | | | 5.68 | 5 | 5 |
| | | diameter | | | | | cm | (5.33 cm) | (5.09 cm) |
| | | of | | | | | | | |
| | | inter | | | | | | | |
| | | node | | | | | | | |
| 5 | 5 | Stem: | | | | | | 4 | 3 |
| | | anthocyanin | | | | | | | |
| | | coloration | | | | | | | |
| | | of | | | | | | | |
| | | inter | | | | | | | |
| | | node | | | | | | | |
| 6 | 6 | Stem: | | | | | | 4 | 2 |
| | | anthocyanin | | | | | | | |
| | | coloration | | | | | | | |
| | | of tip | | | | | | | |
| 7 | 7 | Stem: | | | | | | 4 | 4 |
| | | anthocyanin | | | | | | | |
| | | coloration | | | | | | | |
| | | of inter | | | | | | | |
| | | node | | | | | | | |
| | | and | | | | | | | |
| | | lateral | | | | | | | |
| | | branch | | | | | | | |
| 8 | 8 | Stem: | | | | | | 2 | 1 |
| | | pubescence | | | | | | | |
| | | of tip | | | | | | | |
| 9 | | Leaf | | | | | 139.9 | 5 | 4 |
| | | blade: | | | | | cm² | (148.1 | (120.6 |
| | | size | | | | | | cm²) | cm²) |
| 10 | 9 | Leaf | | | | | | 2 | 2 |
| | | blade: | | | | | | | |
| | | number | | | | | | | |
| | | of lobes | | | | | | | |
| 12 | 11 | Only | | | | | | 5 | 3 |
| | | varieties | | | | | | | |
| | | with | | | | | | | |
| | | leaf | | | | | | | |
| | | blade | | | | | | | |
| | | lobes: | | | | | | | |
| | | Leaf | | | | | | | |
| | | blade: | | | | | | | |
| | | depth | | | | | | | |
| | | of | | | | | | | |
| | | lobing | | | | | | | |
| 13 | 12 | Excluding | | | | | | 2 | 2 |
| | | ornamental | | | | | | | |
| | | variety: | | | | | | | |
| | | Leaf | | | | | | | |
| | | blade: | | | | | | | |
| | | color | | | | | | | |
| | | on | | | | | | | |
| | | upper | | | | | | | |
| | | side | | | | | | | |
| | | (excluding | | | | | | | |
| | | anthocyanin | | | | | | | |
| | | coloration) | | | | | | | |
| 19 | 13 | Excluding | | | | | | 2 | 1 |
| | | ornamental | | | | | | | |
| | | variety: | | | | | | | |
| | | Leaf | | | | | | | |
| | | blade: | | | | | | | |
| | | anthocyanin | | | | | | | |
| | | coloration | | | | | | | |
| | | on | | | | | | | |
| | | upper | | | | | | | |
| | | side | | | | | | | |
| 20 | 14 | Leaf | | very | | | | 1 | 5 |
| | | blade: | | large | | | | | |
| | | extent of | | | | | | | |
| | | anthocyanin | | | | | | | |
| | | coloration | | | | | | | |
| | | abaxial | | | | | | | |
| | | veins | | | | | | | |
| | | on lower | | | | | | | |
| | | side | | | | | | | |
| 21 | 15 | Leaf | | very | | | | 1 | 3 |
| | | blade: | | strong | | | | | |
| | | intensity of | | | | | | | |
| | | anthocyanin | | | | | | | |
| | | coloration | | | | | | | |
| | | abaxial | | | | | | | |
| | | veins on | | | | | | | |
| | | lower | | | | | | | |
| | | side | | | | | | | |
| 22 | | Leaf | | | | | | 1 | 1 |
| | | blade: | | | | | | | |
| | | anthocyanin | | | | | | | |
| | | coloration | | | | | | | |
| | | of nectary | | | | | | | |
| 23 | 16 | Young | light | dark | | | | 7 | 2 |
| | | leaf | brown | brown | | | | | |
| | | blade: | | | | | | | |
| | | main | | | | | | | |
| | | color on | | | | | | | |
| | | upper | | | | | | | |
| | | side | | | | | | | |
| 24 | | Young | light | dark | | | | 5 | 2 |
| | | leaf | brown | brown | | | | | |
| | | blade: | | | | | | | |
| | | main | | | | | | | |
| | | color on | | | | | | | |
| | | lower | | | | | | | |
| | | side | | | | | | | |
| 25 | 17 | Petiole: | | | | | | 3 | 3 |
| | | anthocyanin | | | | | | | |
| | | coloration | | | | | | | |
| 26 | 18 | Petiole: | | | | | 13.1 | 5 | 5 |
| | | length | | | | | cm | (12.5 cm) | (13.4 cm) |
| 29 | 19 | Storage | | | | | | 2 | 3 |
| | | root: | | | | | | | |
| | | shape | | | | | | | |
| 30 | 20 | Storage | | | | | 2.5 | 7 | 5 |
| | | root: | | | | | | (4.7) | (2.7) |
| | | ratio | | | | | | | |
| | | length/ | | | | | | | |
| | | width | | | | | | | |
| 31 | | Storage | | | | | 3.7 | 5 | 5 |
| | | root: | | | | | | (3.9) | (4.7) |
| | | number | | | | | | | |
| 32 | | Storage | | | | | 136 g | 5 | 4 |
| | | root: | | | | | | (183 g) | (143 g) |
| | | mean | | | | | | | |
| | | weight | | | | | | | |
| 33 | 21 | Storage | | | | | 0.050 | 7 | 6 |
| | | root: | | | | | | (0.070) | (0.060) |
| | | thickness | | | | | | | |
| | | of cortex | | | | | | | |
| | | relative | | | | | | | |
| | | to over | | | | | | | |
| | | all | | | | | | | |
| | | diameter | | | | | | | |
| 34 | 22 | Storage | purple | Light | medium | brown | | 10 | 10 |
| | | root: | red | purple | purple | | | | |
| | | main | | | | | | | |
| | | color of | | | | | | | |
| | | skin | | | | | | | |
| 35 | 23 | Storage | brown | | | | | 1 | 1 |
| | | root: | | | | | | | |
| | | secondary | | | | | | | |
| | | color of | | | | | | | |
| | | skin | | | | | | | |
| 36 | 24 | Storage | | | | | | 5 | 5 |
| | | root: | | | | | | | |
| | | main | | | | | | | |
| | | color of | | | | | | | |
| | | flesh | | | | | | | |
| 37 | 25 | Storage | | | | | | 3 | 3 |
| | | root: | | | | | | | |
| | | intensity | | | | | | | |
| | | of main | | | | | | | |
| | | color of | | | | | | | |
| | | flesh | | | | | | | |
| 38 | 26 | Storage | purple | | | | — | 7 | 7 |
| | | root: | | | | | | | |
| | | secondary | | | | | | | |
| | | color of | | | | | | | |
| | | flesh | | | | | | | |
| 39 | 27 | Storage | | | | | | 3 | 1 |
| | | root: | | | | | | | |
| | | depth | | | | | | | |
| | | of eyes | | | | | | | |
| 40 | | Storage | light | orange | light | purple | | 11 | 11 |
| | | root: | orange | | purple | | | | |
| | | color of | | | | | | | |
| | | steamed | | | | | | | |
| | | flesh | | | | | | | |
| 44 | | Southern | | | | | | 3 | 4 |
| | | rootknot | | | | | | | |
| | | nematode | | | | | | | |
| | | (Meloisogyne) | | | | | | | |
| | | resistance | | | | | | | |

TABLE 3
Description of physical characteristics of ‘Churakanasa’ and values for
characteristics of similar varieties for year 2019

| | | | Class values of candidate variety (Comparison with | | | | | | |
|---|---|---|---|---|---|---|---|---|---|
| Characteristics | UPOV | | Ex.Var.(standard varieties)) ‘CHURAKANASA’ | | | | | | |
| No. | No. | Characteristics | 01 | 02 | 03 | 04 | 05 | 06 | 07 |
| 1 | 1 | Plant: | upright | | semi- | | spreading | | |
| | | growth | | | upright | | | | |
| | | habit | | | | | | | |
| 2 | 2 | Stem: | | | short | | medium | | long |
| | | length | | | | | | | |
| | | of | | | | | | | |
| | | primary | | | | | | | |
| | | shoots | | | | | | | |
| 3 | 3 | Stem: | | | short | | medium | | long |
| | | length | | | | | | | |
| | | of | | | | | | | |
| | | inter | | | | | | | |
| | | node | | | | | | | |
| 4 | 4 | Stem: | | | small | | medium | Medium | large |
| | | diameter | | | | | | to | |
| | | of | | | | | | large | |
| | | inter | | | | | | | |
| | | node | | | | | | | |
| 5 | 5 | Stem: | absent | weak | medium | strong | | | |
| | | anthocyanin | or | | | | | | |
| | | coloration | very | | | | | | |
| | | of | weak | | | | | | |
| | | inter | | | | | | | |
| | | node | | | | | | | |
| 6 | 6 | Stem: | absent | weak | medium | strong | | | |
| | | anthocyanin | or | | | | | | |
| | | coloration | very | | | | | | |
| | | of tip | weak | | | | | | |
| 7 | 7 | Stem: | absent | weak | medium | strong | | | |
| | | anthocyanin | or | | | | | | |
| | | coloration | very | | | | | | |
| | | of | weak | | | | | | |
| | | node | | | | | | | |
| | | and | | | | | | | |
| | | lateral | | | | | | | |
| | | branch | | | | | | | |
| 8 | 8 | Stem: | absent | sparce | medium | dense | | | |
| | | pubescence | or | | | | | | |
| | | of tip | very | | | | | | |
| | | | sparce | | | | | | |
| 9 | | Leaf | | | small | | medium | Medium | large |
| | | blade: | | | | | | to | |
| | | size | | | | | | large | |
| 10 | 9 | Leaf | absent | Three | five | seven | | | |
| | | blade: | | lobes | lobes | lobes | | | |
| | | number | | | | | | | |
| | | of lobes | | | | | | | |
| 12 | 11 | Only | | | shallow | | moderate | | deep |
| | | varieties | | | | | | | |
| | | with | | | | | | | |
| | | leaf | | | | | | | |
| | | blade | | | | | | | |
| | | lobes: | | | | | | | |
| | | Leaf | | | | | | | |
| | | blade: | | | | | | | |
| | | depth | | | | | | | |
| | | of | | | | | | | |
| | | lobing | | | | | | | |
| 13 | 12 | Excluding | yellow | green | grey | | | | |
| | | ornamental | green | | green | | | | |
| | | variety: | | | | | | | |
| | | Leaf | | | | | | | |
| | | blade: | | | | | | | |
| | | color | | | | | | | |
| | | on | | | | | | | |
| | | upper | | | | | | | |
| | | side | | | | | | | |
| | | (excluding | | | | | | | |
| | | anthocyanin | | | | | | | |
| | | coloration) | | | | | | | |
| 19 | 13 | Excluding | absent | weak | medium | strong | | | |
| | | ornamental | or | | | | | | |
| | | variety: | very | | | | | | |
| | | Leaf | weak | | | | | | |
| | | blade: | | | | | | | |
| | | anthocyanin | | | | | | | |
| | | coloration | | | | | | | |
| | | on | | | | | | | |
| | | upper | | | | | | | |
| | | side | | | | | | | |
| 20 | 14 | Leaf | absent | | small | | medium | | large |
| | | blade: | or | | | | | | |
| | | extent of | very | | | | | | |
| | | anthocyanin | small | | | | | | |
| | | coloration | | | | | | | |
| | | abaxial | | | | | | | |
| | | veins | | | | | | | |
| | | on lower | | | | | | | |
| | | side | | | | | | | |
| 21 | 15 | Leaf | very | | weak | | medium | | strong |
| | | blade: | weak | | | | | | |
| | | intensity | | | | | | | |
| | | of | | | | | | | |
| | | anthocyanin | | | | | | | |
| | | coloration | | | | | | | |
| | | abaxial | | | | | | | |
| | | veins on | | | | | | | |
| | | lower | | | | | | | |
| | | side | | | | | | | |
| 22 | | Leaf | absent | | weak | | medium | | strong |
| | | blade: | or | | | | | | |
| | | anthocyanin | very | | | | | | |
| | | coloration | weak | | | | | | |
| | | of nectary | | | | | | | |
| 23 | 16 | Young | yellow | light | medium | dark | light | medium | Purplish |
| | | leaf | green | green | green | green | purple | purple | brown |
| | | blade: | | | | | | | |
| | | main | | | | | | | |
| | | color on | | | | | | | |
| | | upper | | | | | | | |
| | | side | | | | | | | |
| 24 | | Young | yellow | light | medium | dark | Light | medium | purplish |
| | | leaf | green | green | green | green | purple | purple | brown |
| | | blade: | | | | | | | |
| | | main | | | | | | | |
| | | color on | | | | | | | |
| | | lower | | | | | | | |
| | | side | | | | | | | |
| 25 | 17 | Petiole: | absent | | weak | | medium | | strong |
| | | anthocyanin | or | | | | | | |
| | | coloration | very | | | | | | |
| | | | weak | | | | | | |
| 26 | 18 | Petiole: | | | short | | medium | | long |
| | | length | | | | | | | |
| 29 | 19 | Storage | obovate | elliptic | ovate | oblong | irregular | | |
| | | root: | | | | | | | |
| | | shape | | | | | | | |
| 30 | 20 | Storage | | | moderately | | medium | Medium | moderately |
| | | root: | | | compressed | | | to | elongated |
| | | ratio | | | | | | moderately | |
| | | length/ | | | | | | elongated | |
| | | width | | | | | | | |
| 31 | | Storage | | | few | | medium | | many |
| | | root: | | | | | | | |
| | | number | | | | | | | |
| 32 | | Storage | | | small | | medium | | large |
| | | root: | | | | | | | |
| | | mean | | | | | | | |
| | | weight | | | | | | | |
| 33 | 21 | Storage | | | thin | | medium | | thick |
| | | root: | | | | | | | |
| | | thickness | | | | | | | |
| | | of cortex | | | | | | | |
| | | relative | | | | | | | |
| | | to over | | | | | | | |
| | | all | | | | | | | |
| | | diameter | | | | | | | |
| 34 | 22 | Storage | white | light | yellow | orange | brownish | pink | red |
| | | root: | | beige | | | orange | | |
| | | main | | | | | | | |
| | | color of | | | | | | | |
| | | skin | | | | | | | |
| 35 | 23 | Storage | absent | white | yellow | orange | pink | red | purple |
| | | root: | | | | | | | |
| | | secondary | | | | | | | |
| | | color of | | | | | | | |
| | | skin | | | | | | | |
| 36 | 24 | Storage | white | light | yellow | orange | purple | | |
| | | root: | | beige | | | | | |
| | | main | | | | | | | |
| | | color of | | | | | | | |
| | | flesh | | | | | | | |
| 37 | 25 | Storage | light | medium | dark | | | | |
| | | root: | | | | | | | |
| | | intensity | | | | | | | |
| | | of main | | | | | | | |
| | | color of | | | | | | | |
| | | flesh | | | | | | | |
| 38 | 26 | Storage | white | light | yellow | orange | pink | red | red- |
| | | root: | | beige | | | | | purple |
| | | secondary | | | | | | | |
| | | color of | | | | | | | |
| | | flesh | | | | | | | |
| 39 | 27 | Storage | shallow | medium | deep | | | | |
| | | root: | | | | | | | |
| | | depth | | | | | | | |
| | | of eyes | | | | | | | |
| 40 | | Storage | white | gray | light | light | light | yellow | gray |
| | | root: | | white | yellow | beige | yellow | | yellow |
| | | color of | | | white | | | | |
| | | steamed | | | | | | | |
| | | flesh | | | | | | | |
| 44 | | Southern | weak | moderately | medium | moderately | strong | | |
| | | rootknot | | weak | | strong | | | |
| | | nematode | | | | | | | |
| | | (Meloisogyne) | | | | | | | |
| | | resistance | | | | | | | |
| | | | Class values of candidate variety | | | | | | |
| | | | (Comparison with Ex.Var.(standard | | | | Remarks | Class Values of | |
| Characteristics | UPOV | | varieties)) ‘CHURAKANASA’ | | | | (Measurement | most similar varieties | |
| No. | No. | Characteristics | 08 | 09 | 10 | 11 | Value etc.) | ‘Ayamurasaki’ | ‘Murasakimasari’ |
| 1 | 1 | Plant: | | | | | | 5 | 5 |
| | | growth | | | | | | | |
| | | habit | | | | | | | |
| 2 | 2 | Stem: | | | | | 131.8 | 5 | 5 |
| | | length | | | | | cm | (147.7 cm) | (147.0 cm) |
| | | of | | | | | | | |
| | | primary | | | | | | | |
| | | shoots | | | | | | | |
| 3 | 3 | Stem: | | | | | 6.7 | 5 | 5 |
| | | length | | | | | cm | (6.0 cm) | (7.3 cm) |
| | | of | | | | | | | |
| | | inter | | | | | | | |
| | | node | | | | | | | |
| 4 | 4 | Stem: | | | | | 5.68 | 5 | 5 |
| | | diameter | | | | | cm | (5.33 cm) | (5.09 cm) |
| | | of | | | | | | | |
| | | inter | | | | | | | |
| | | node | | | | | | | |
| 5 | 5 | Stem: | | | | | | 4 | 3 |
| | | anthocyanin | | | | | | | |
| | | coloration | | | | | | | |
| | | of | | | | | | | |
| | | inter | | | | | | | |
| | | node | | | | | | | |
| 6 | 6 | Stem: | | | | | | 4 | 2 |
| | | anthocyanin | | | | | | | |
| | | coloration | | | | | | | |
| | | of tip | | | | | | | |
| 7 | 7 | Stem: | | | | | | 4 | 4 |
| | | anthocyanin | | | | | | | |
| | | coloration | | | | | | | |
| | | of inter | | | | | | | |
| | | node | | | | | | | |
| | | and | | | | | | | |
| | | lateral | | | | | | | |
| | | branch | | | | | | | |
| 8 | 8 | Stem: | | | | | | 2 | 2 |
| | | pubescence | | | | | | | |
| | | of tip | | | | | | | |
| 9 | | Leaf | | | | | 215.2 | 5 | 4 |
| | | blade: | | | | | cm² | (190.0 cm²) | (182.5 cm²) |
| | | size | | | | | | | |
| 10 | 9 | Leaf | | | | | | 2 | 2 |
| | | blade: | | | | | | | |
| | | number | | | | | | | |
| | | of lobes | | | | | | | |
| 12 | 11 | Only | | | | | | 5 | 3 |
| | | varieties | | | | | | | |
| | | with | | | | | | | |
| | | leaf | | | | | | | |
| | | blade | | | | | | | |
| | | lobes: | | | | | | | |
| | | Leaf | | | | | | | |
| | | blade: | | | | | | | |
| | | depth | | | | | | | |
| | | of | | | | | | | |
| | | lobing | | | | | | | |
| 13 | 12 | Excluding | | | | | | 2 | 2 |
| | | ornamental | | | | | | | |
| | | variety: | | | | | | | |
| | | Leaf | | | | | | | |
| | | blade: | | | | | | | |
| | | color | | | | | | | |
| | | on | | | | | | | |
| | | upper | | | | | | | |
| | | side | | | | | | | |
| | | (excluding | | | | | | | |
| | | anthocyanin | | | | | | | |
| | | coloration) | | | | | | | |
| 19 | 13 | Excluding | | | | | | 2 | 1 |
| | | ornamental | | | | | | | |
| | | variety: | | | | | | | |
| | | Leaf | | | | | | | |
| | | blade: | | | | | | | |
| | | anthocyanin | | | | | | | |
| | | coloration | | | | | | | |
| | | on | | | | | | | |
| | | upper | | | | | | | |
| | | side | | | | | | | |
| 20 | 14 | Leaf | | very | | | | 1 | 5 |
| | | blade: | | large | | | | | |
| | | extent of | | | | | | | |
| | | anthocyanin | | | | | | | |
| | | coloration | | | | | | | |
| | | abaxial | | | | | | | |
| | | veins | | | | | | | |
| | | on lower | | | | | | | |
| | | side | | | | | | | |
| 21 | 15 | Leaf | | very | | | | 1 | 3 |
| | | blade: | | strong | | | | | |
| | | intensity of | | | | | | | |
| | | anthocyanin | | | | | | | |
| | | coloration | | | | | | | |
| | | abaxial | | | | | | | |
| | | veins on | | | | | | | |
| | | lower | | | | | | | |
| | | side | | | | | | | |
| 22 | | Leaf | | | | | | 1 | 1 |
| | | blade: | | | | | | | |
| | | anthocyanin | | | | | | | |
| | | coloration | | | | | | | |
| | | of nectary | | | | | | | |
| 23 | 16 | Young | light | dark | | | | 7 | 2 |
| | | leaf | brown | brown | | | | | |
| | | blade: | | | | | | | |
| | | main | | | | | | | |
| | | color on | | | | | | | |
| | | upper | | | | | | | |
| | | side | | | | | | | |
| 24 | | Young | light | dark | | | | 5 | 2 |
| | | leaf | brown | brown | | | | | |
| | | blade: | | | | | | | |
| | | main | | | | | | | |
| | | color on | | | | | | | |
| | | lower | | | | | | | |
| | | side | | | | | | | |
| 25 | 17 | Petiole: | | | | | | 3 | 3 |
| | | anthocyanin | | | | | | | |
| | | coloration | | | | | | | |
| 26 | 18 | Petiole: | | | | | 25.3 | 5 | 5 |
| | | length | | | | | cm | (24.3 cm) | (25.7 cm) |
| 29 | 19 | Storage | | | | | | 2 | 3 |
| | | root: | | | | | | | |
| | | shape | | | | | | | |
| 30 | 20 | Storage | | | | | 3.4 | 7 | 6 |
| | | root: | | | | | | (4.9) | (3.5) |
| | | ratio | | | | | | | |
| | | length/ | | | | | | | |
| | | width | | | | | | | |
| 31 | | Storage | | | | | 3.3 | 6 | 6 |
| | | root: | | | | | | (5.0) | (5.9) |
| | | number | | | | | | | |
| 32 | | Storage | | | | | 142 g | 6 | 5 |
| | | root: | | | | | | (205 g) | (160 g) |
| | | mean | | | | | | | |
| | | weight | | | | | | | |
| 33 | 21 | Storage | | | | | 0.075 | 7 | 6 |
| | | root: | | | | | | (0.072) | (0.064) |
| | | thickness | | | | | | | |
| | | of cortex | | | | | | | |
| | | relative | | | | | | | |
| | | to over | | | | | | | |
| | | all | | | | | | | |
| | | diameter | | | | | | | |
| 34 | 22 | Storage | purple | Light | medium | brown | | 10 | 10 |
| | | root: | red | purple | purple | | | | |
| | | main | | | | | | | |
| | | color of | | | | | | | |
| | | skin | | | | | | | |
| 35 | 23 | Storage | brown | | | | | 1 | 1 |
| | | root: | | | | | | | |
| | | second- | | | | | | | |
| | | ary | | | | | | | |
| | | color of | | | | | | | |
| | | skin | | | | | | | |
| 36 | 24 | Storage | | | | | | 5 | 5 |
| | | root: | | | | | | | |
| | | main | | | | | | | |
| | | color of | | | | | | | |
| | | flesh | | | | | | | |
| 37 | 25 | Storage | | | | | | 3 | 3 |
| | | root: | | | | | | | |
| | | intensity | | | | | | | |
| | | of main | | | | | | | |
| | | color of | | | | | | | |
| | | flesh | | | | | | | |
| 38 | 26 | Storage | purple | | | | — | 7 | 7 |
| | | root: | | | | | | | |
| | | secondary | | | | | | | |
| | | color of | | | | | | | |
| | | flesh | | | | | | | |
| 39 | 27 | Storage | | | | | | 3 | 1 |
| | | root: | | | | | | | |
| | | depth | | | | | | | |
| | | of eyes | | | | | | | |
| 40 | | Storage | light | orange | light | purple | | 11 | 11 |
| | | root: | orange | | purple | | | | |
| | | color of | | | | | | | |
| | | steamed | | | | | | | |
| | | flesh | | | | | | | |
| 44 | | Southern | | | | | | 3 | 4 |
| | | rootknot | | | | | | | |
| | | nematode | | | | | | | |
| | | (Meloisogyne) | | | | | | | |
| | | resistance | | | | | | | |

The sprouting trait (same as sprouting habit) of ‘Churakanasa’ is ‘poor’ and lower than that of ‘Ayamurasaki ’ and ‘Murasakimasari’. The extent and the intensity of anthocyanin coloration on abaxial veins on lower side of the leaf blade of ‘Churakanasa’ are the same as that of ‘Murasakimasari’ and are larger and stronger than those of ‘Ayamurasaki’. The color on upper side of young leaf of ‘Churakanasa’ is ‘purplish brown’, which is the same as ‘Ayamurasaki’ and different from ‘Murasakimasari’. The strength of the storage root's neck of ‘Churakanasa’ is ‘medium’. The shape of storage root of ‘Churakanasa’ is ‘elliptic’ and ‘spindle’. The skin color of storage root of ‘Churakanasa’ is ‘purple’, which is lighter than that of ‘Ayamurasaki’. The appearance of ‘Churakanasa’ is ‘medium’. There is no groove, ridge and dehiscence on the storage root of ‘Churakanasa’.

Compared to ‘Ayamurasaki’ and ‘Murasakimasari’, in standard cultivation, the mean weight of the good quality potatoes (storage roots) and the total weight of the good quality storage roots of ‘Churakanasa’ are lower. The mean weight of storage roots of ‘Churakanasa’ is 183 kg/a, while the mean weight of storage roots is 329 kg/a for ‘Murasakimasari’ and 307 kg/a for ‘Ayamurasaki.’ Also under earlier harvest condition, the mean weight of the good quality storage roots and the number of good quality storage roots per one plant of ‘Churakanasa’ are low, and the total weight of the good quality storage roots of ‘Churakanasa’ is lower than that of ‘Ayamurasaki’. The number of good quality storage roots per one plant of ‘Churakanasa’ is low, and the mean weight of the good quality storage roots of ‘Churakanasa’ is a little light, too. Therefore the total weight of the good quality storage roots of ‘Churakanasa’ is lower than that of ‘Purple Sweet Lord.’

The steamed flesh color of ‘Churakanasa’ is ‘purple’, quality of flesh in standard cultivation is ‘a little crumbly’ and the taste of flesh is ‘medium’, which exceeds that of ‘Ayamurasaki ’ and ‘Murasakimasari’. The color value of anthocyanin of ‘Churakanasa’ is a little higher than that of ‘Churakoibeni’, in the breeding place. The color value of anthocyanin of ‘Churakanasa’ is lower than that of ‘Ayamurasaki’. In the breeding place, the color of paste of ‘Churakanasa’ is a little more bluish than that of ‘Churakoibeni’ (the value b* of ‘Churakanasa’ is a little smaller). The variable b* is commonly used to represent color difference. The higher the value of b*, the more yellowish the represented color, and the lower the value of b*, the more bluish the represented color.

The paste made from the flesh of ‘Churakanasa’ is different from ‘Churakoibeni’ in color tone and the paste is moderately harder than that of ‘Churakoibeni’, but there is no particular problem associated with this hardness. It is easy to peel and process. In addition, the flavor of ‘Churakanasa’ is better than ‘Churakoibeni’, and it is suitable for processed products. After preparation of the paste, Beniimo tarts were prepared with use of secondary material according to a standard recipe, and sensory evaluation was conducted. The difference in the color tone of ‘Churakanasa’ and ‘Churakoibeni’ is such that it can only be understood by comparing them side by side, and there is no problem. The paste of ‘Churakanasa’ tends to become ‘lumpy’ during processing, but that is no problem. Compared to ‘Churakoibeni’, it has better sweet potato flavor and taste.

Storability of storage root of ‘Churakanasa’ in storage house and laboratory is ‘moderately difficult’ and worse than that of ‘Ayamurasaki’. Southern root-knot nematode (M incognita) resistance of ‘Churakanasa’ is ‘medium’ and the resistance to Minami root-lesion nematodes (P. coffeae) is ‘moderately strong’. In characterization tests, the soil rot resistance of ‘Churakanasa’ was judged to be ‘moderately strong’. For the soil rot resistance test, the test site was naturally infested fields in Okinawa. Fertilization condition was N: P₂O₅: K₂O=0.45:0.45:0.90 (kg/a). Cultivation conditions were mulching-free cultivation, 80 cm ridges interval×30cm strains interval, 5 strains/block×3 repeats. Planting was done on Apr. 28, 2014 with excavation on Sep. 29, 2014. Additional planting was done on May 18, 2015 with excavation on Oct. 9, 2015. Additional planting was also done on Jun. 8, 2016 with excavation on Nov. 18, 2016. Damage investigation involved dividing into stems and storage roots at harvesting, counting the blotches respectively, and then converting them into an evaluation value. The attitude evaluation on a scale of one to six was carried out. The stem or storage root having no blotch is evaluation value ‘1’, having 1 to 3 blotches is evaluation value ‘2’, having 4 to 6 blotches is evaluation value ‘3’, having 7 to 9 blotches is evaluation value ‘4’, having 10 or more blotches is evaluation value ‘5’ and blighted strain is evaluated at ‘6’. Judgement criteria based on this was that an evaluation value less than ‘2’ is ‘strong’, ‘2’ to less than ‘3’ is ‘moderately strong’, ‘3’ to less than ‘4’ is ‘moderately weak’, and evaluation value of 4 or more than ‘4’ is ‘weak’. The soil rot resistance of ‘Churakanasa’ was found to be ‘moderately strong.’ In the characterization tests, the resistance of ‘Churakanasa’ to black rot disease was judged to be ‘medium’. In other tests, the resistance of ‘Churakanasa’ to Southern root-knot nematode (M. incognita) is ‘moderately weak’, the soil rot resistance of ‘Churakanasa’ is ‘medium’, and the resistance of ‘Churakanasa’ to stem rot (Fusarium oxysporum f sp. batatas) is ‘medium’.

Tests for resistance to weevils were also carried out. The test site was a field in Okinawa. Fertilization conditions were N: P₂O₅: K₂O=0.45:0.45:0.90 (kg/a). Cultivation conditions were mulching-free cultivation, 80 cm ridges interval×30 cm strains interval, 10 strains/block×3 repeats. Planting was done on May 13, 2016 with excavation on Oct. 25, 2016. Additional planting was done on May 19, 2017 with excavation on Nov. 10, 2017. Additional planting was also done on May 11, 2018 with excavation on Oct. 29, 2018. About 2-3 months after planting, 40 piglets (Euscepes postfasciatus) were left per block to investigate the harvested material. Cylas formicarius were left to spontaneous invasion. ‘Churakanasa’ is determined to be damaged if there is at least one insect damage mark on the tuber (storage root) cross section sliced to 3 mm thickness. A sound plant without insect damage mark on the tuber cross section sliced to 3 mm thickness is called stage “1”, if insect damage mark is less than 5% of the cross section the stage is “2”, if insect damage mark is 5% or more to less than 30% of the cross section the stage is “3”, if insect damage mark is 30% or more to less than 50% of the cross section the stage is “4”, and if insect damage mark is 50% or more of the cross section the stage is “5”, the attitude evaluation of the five stages was carried out. Judgement was based on “damage rate”: 10 is considered “strong”; 11 to 30 is considered “slightly strong”; 31 to 50 is considered “medium”; 51 to 70 is considered “slightly weak”, 71 and higher is considered “weak”. The damage degree and the damage rate of ‘Churakanasa’ to the weevils tend to be lower than those of existing varieties in the same field, and the total yield of ‘Churakanasa’ including the damaged storage roots shows a high level even in the presence of the weevils.

‘Churakanasa’ is not suitable for cultivation in Kyushu because the yield (total weight) of the good quality storage roots is small and the sprouting quality is poor at the cultivation site (Miyakonojo City, Miyazaki Prefecture).

It will be understood that the average size of the plant and fruit may vary with location, season, nutrition, irrigation, etc.

What is claimed is:

1. A new and distinct Ipomoea batatas plant named ‘Churakanasa’ as illustrated and described.
